(19) United States
(12) Patent Application Publication
(10) Pub. No.: US 2008/0198328 A1
(43) Pub. Date: Aug. 21, 2008

(54) System and method self enabling customers to obtain refraction specifications for, and purchase of, previous or new fitted eyeglasses

(76) Inventors: Joseph S. Seriani, Shinnston, WV (US); Hal E. Wilson, Raleigh, NC (US)

Correspondence Address: JOSEPH S. SERIANI, 69 WALNUT ST., SHINNSTON, WV, 26431, US

(21) Appl. No.: 11/707,237

(22) Filed: Feb. 16, 2007

(51) Int. Cl. A61B 3/10

(52) U.S. Cl. 351/205

(57) ABSTRACT

A system and method for obtaining a sight screening as well as previous and current optical prescriptions (specifications) for virtual try-on and purchase of glasses and contact lenses operated by one's self, through a voice-activated response self-refraction, or a technician/optician assisted refraction, at a convenient location or possibly several different customer diagnostic, electronic store locations where digital instruments interface and may transfer either directly back to the customer, or to a remote, interactive audio-video diagnosing and assisting, consulting eye doctor (ophthalmologist or optometrist) who interprets the digital data, diagnoses, consults, delivers health care, and authorizes the specifications, whereby the store technician/optician gathers data for purchase and electronically sends the data to a lens lab for fabrication and delivery of glasses or contact lenses to the store or directly to the customer; and, the customer can purchase selected glasses or contact lenses from any internet PC address, whereby the technician provides the customer with a CD containing customer data and digital imaging for reorders either through a PC or a selected retail location.

### BACKGROUND OF THE INVENTION

1. Technical Field

Specifically, the invention of the above application is an improved system employing digital data derived from integrating technologically advanced ophthalmic instruments and digital imaging devices. It provides a more efficient method for customers to obtain optical specifications heretofore only available by an examiner who provides refractive results obtained through a traditional, office-visit refraction. It uses a highly efficient system of subjective and objective refractive optical instruments integrated into a customer voice-activated response that provides a current optical prescription as precise and accurate, if not better, that had the examination been performed by an experienced clinician. The invention is virtually a do-it-yourself accurate optical prescription.

The efficiency, speed, and do-it-yourself-refraction aspects of this method are designed to save customers money and time. Beforehand, this process was much costlier and purchased at an optometrist or optician office and typically performed by a skilled technician—the skilled examiner or skilled staff technician, and with no interfacing or ability to gather data necessary to provide refraction and glasses for immediate purchase either at a convenient retail location or on the internet. It also provides inexpensive and efficient screenings to determine whether visual loss is merely refractive or due to a medical anomaly which in that case the customer is referred to an ophthalmologist who typically does not sell or dispense eye glasses.

In addition, the invention is an improved and more efficient digital imaging system that allows the customer to select and purchase frames and lenses based on the previous or current optical prescription self-derived as described above. It is a one-stop-shop, never before available or offered to the public in such time and cost saving fashion.

The present invention pertains to the field of vision and vision corrective devices—eyeglasses. Specifically, the present invention is concerned with determining specifications of eyeglasses, visual acuity and refractive error for determining specifications or prescription for optical lenses, and determining information and fit for glasses or contact lenses to a customer through obtaining digital images and information of a customer. This data is sent to an eye doctor, or pre-programmed computerized system for interpretation, results, and/or electronic signature results. The customer also selects and may purchase the prescription, eyeglasses or contact lenses either through a PC or at a convenient location.

2. Description of Related Art

As the internet has become increasingly useful, more emphasis has been placed to enabling customers to purchase medical devices through digital images of those devices on PC's. Internet eyeglass and contact lens shopping is rapidly increasing. This has directly resulted in systems and applications in which a PC adjusts the size of a product and, using a predetermined image of the customer, shows the customer how the customer would appear wearing the product or device.

In particular, U.S. patent Ser. No. 08/874,269 to Fay is directed to a system that shows a customer how the customer's frame would appear on a PC so that customers with pass codes are suggested basic frames which are then sent to opticians, optical retail locations, who then send frames to optical labs who then send the frame with lenses back to opticians retail locations who then ask the customer to return for dispensing. In this system, customers are unable to have a complete pair of eyeglasses or contact lenses sent directly to customers. It does ascertain information about the customer's head and face shape from among seven basic shapes, hair color, skin tone, and sex, making it available on PC's at store locations or elsewhere suggesting predetermined eyeglass frames, structural material, style, and frame subsets to match one of the seven facial shapes. In this previous art, the customer is prompted either to agree or to choose a different basic shape but unable to obtain a prescription, or obtain a complete pair of glasses or contacts.

U.S. Pat. No. 5,280,570 to Jordan is directed to a system that shows a customer how the customer's eyes would appear to another person because of wearing glasses with a certain specifications, causing the customer's eyes to appear larger or smaller, depending on the specifications, than without the lenses. This particular system includes components that permit a customer to try on various eyeglasses, observe how the customer would appear wearing the glasses, and place an order. These systems use lifestyle information obtained from the customer to limit what models of frames it merchandises to the customer, either physically collocated in a store where the customer must go in person or on a PC to electronically try on frames and to place an order. Such systems make no mention of obtaining the actual lens specifications or prescriptions and are of no use in determining the specifications and fit of lenses, nor do they deploy digital imaging or information to concurrently determine visual acuity with previous glasses, specifications of previous glasses, current refractive error, and derive a complete means to transact, merchandise, and offer for purchase current prescription eyeglasses and contact lenses to a customer either at a convenient location or at a remote PC.

U.S. Pat. No. 5,515,268 to Youda discusses a system where a customer selects a product at a store location from a product list stored in a PC at a remote location. In this system a sensor detects customer size information and transmits it to the remote PC. The remote PC then selects a product of appropriate size and shape and sends an image to the store for display on a monitor which shows the product superimposed on the customer. The product is fitted to the customer's body image using various PC transformations. This invention is not directed to a particular product. It instructs the use of an actuator loop which transfers product information to the customer. Such an actuator is of no use in fitting glasses or contacts or determining optical specifications.

U.S. Pat. No. 5,592,258 of Norton et al. utilizes a method to provide a fit for eyeglasses to a customer by taking pictures of the customer at one location and transmitting those pictures to a remote location where the pictures are evaluated to allow an accurate determination of the shape and size of the customer's head for custom making of eyeglass frames. Norton, however, does not teach or show the customer how the customer would appear in alternative or different pairs of eyeglass frames, nor does he derive visual acuity and refractive error to determine the lens specifications.

In all of this prior art, the customer either places an order at a storefront or at a PC after trying on frames. In Youda's and Fay's cases, the orders are transmitted to a remote PC, the frames are either ordered at a store front or on a PC, however, no information to determine lens specifications that may be fit for eyeglass frames or contact lenses.

What is needed is a way to enable a customer to derive and determine their own specifications or refractive error or lens prescriptions for eyeglasses or contact lenses. What is needed is more than a way of enabling a customer to conveniently try on different eyeglass frames and order at a convenient retail location or at home on a PC because without a way to self-determine accurate customer's lens specifications or derive signatures from eye doctors, to confirm, interpret, and provide specifications for eyeglasses or contact lenses, an order for such can not be filled or sold. Thus, with present art, customers are obligated to obtain separately and elsewhere eye examinations, sight screenings, or refractive errors for lens specifications and fitting from independent eye doctors, not mentioned or involved in the system and obviously located elsewhere in previous art. Even after questioning a customer for lifestyle and other information and showing how the customer will appear in predetermined frames, in all of the other art, choices of different eyeglass frames must be sent to opticians who fill specifications and fit glasses by photographs or frame images of the how-the-customer-appears with predetermined frames. Mention of prescription lenses, or clear glasses, or sunglasses in prior art merely states the obvious that frames purchased in previous systems of prior art, for example Fay's, is that selected frames may house prescription lenses, and in Fay's, the customers must additionally seek and select both an eye doctor to derive a prescription, and an optician to fill and fit the prescription with predetermined, suggested frames or by the customer's own camera.

For example, customers often determine which prescription lens specifications for reading glasses when they shop places that sell eyeglasses for reading by trying on different eyeglasses themselves and merely surmising what lens specifications from which they seem to see the best to purchase. Those eyeglass frames body prescription lenses that are the same for both eyes and do not account for astigmatic, cylindrical refractive error. With present art, specifications for prescription lenses are determined accurately and take into account refractive errors separate and independent for each eye, as well as refractive errors for spherical power, cylindrical power, and axis degree determination, not possible with pre-made or molded prescription lenses in reading eyeglasses. So, for example, present art takes the guess work out of purchasing reading glasses or readers, as they are called and provides a more accurate lens and specifications determination so that the customer adapts more comfortably and has better vision. Another novel use of the present art is that the customer may not have to obtain a complete eye examination from an eye doctor in order to just obtain new specifications for lenses for eyeglasses or contact lenses. An eye doctor usually performs about twenty tests or procedures; most of them are medical and used to make medical and eye health diagnoses. Included in these procedures is the one, usually subjective, test called a refraction which primarily determines current or new specifications for eyeglass fabrication. With present art it is not necessary to have to go through the entire process of medical-eye examination when the customer wants to purchase new specifications or refraction specifications. This would be particularly useful when the customer has broken or loses his eyeglasses and needs to conveniently and quickly purchase new ones.

Previous art diagnoses and suggests predetermined, frames and frame characteristics. What is not obvious and needed is art that diagnoses and determines current optical prescriptions for specifications from eye doctors, either conveniently located near customers or retail locations or electronically on-line, and through electronic-remote authorize the specifications or prescription which is immediately able to be filled by opticians conveniently located near the customer or retail locations or electronically on-line, and present means for customers to purchase vision screeners and exams, refractions, and a complete pair of eyeglasses and contact lenses, rather than just apparel frames. In present art, opticians take weeks to sell, process, and fill orders. What is needed is art that uses a system that takes about a day to sell, process, and fill orders.

### SUMMARY OF INVENTION

The present invention is a means and method for providing customers with their visual acuities, refractive errors, and specifications or prescriptions for glasses and contacts, and the means, simultaneously or at a later time, to use those lens specifications in conjunction with the selection of eyeglasses or contacts, either at a convenient store or at a PC elsewhere, and to choose and purchase a complete pair of eyeglasses or contact lenses.

In one preferred embodiment the method requires that the customer visit any of a number of possible locations, called optical diagnostic and retail outlets, interfaced to electronically ascertain information about the customer's present glasses specifications, visual acuity, refractive error or optical prescription or lens lab specifications, and through digital imaging offer a complete fit of eyeglasses through selections of frames and lenses superimposed on images viewable on a PC, either at the optical diagnostic location, or elsewhere on a PC. Each of these diagnostic locations is connected to remote electronic eye doctors who review, interpret, and authorize sufficient information for purchase and selection of eyeglasses and contacts by the customer. This information is ascertained by integrated, electronic-instrument systems utilizing auto-refractometers, auto-lensometers, auto-phoropters, and other diagnostic optical instruments, that store and send data over a protected server to PC's. The information is sufficient for a customer to purchase refractions and eyeglasses. The fabrication data is then immediately sent to a lab via the internet which either sends the eyeglasses or contact lenses directly to the customer or to the diagnostic location.

A further object of the present invention is to make it convenient for customers to purchase refractions, eyeglasses, and contact lenses and assist them with information and digital images necessary to make purchase choices either at a diagnostic location or elsewhere on a PC.

The above objects and others not specifically recited are realized in a specific illustrative embodiment of a system enabling customers to obtain sufficient information to purchase a complete pair of eyeglasses or contact lenses either at a diagnostic location or elsewhere on a PC. In addition, this information can be retrieved and later modified by customers on PC's or at the diagnostic location.

The present invention is unlike any other vision screening, with or without an unskilled technician, and accurately determines whether one may have a suspicious vision disorder, anomaly or disease, as well as provide new prescription eyeglasses in a unique way. There has never been an efficient system utilizing a unique integration of electronic instruments, interfaced in a way to obtain a complete prescription for, and fitting and purchasing of, new eyeglasses in about ten minutes, and an improvement of about five-times more access to those who need vision correction. Prior art systems and methods do not have the ability to improve the sight of nearly 1 Billion people worldwide, by US standards, who are legally blind. The present invention will materially enhance the quality of life of human kind by contributing to the accuracy, accessibility, and affordability of prescription eyeglasses. This system will improve the vision of people who need corrective lenses.

### DESCRIPTION

### Brief Description of Drawing

The above and other objects, features and advantages of the invention are described in a flow diagram illustration of procedures and description which will become apparent from a consideration of the subsequent detailed description presented in connection with accompanying drawings, in which the elements of a system to obtain a visual acuity screening, or refraction, or prescription lens specifications for current and/or previous eyeglasses; to concurrently obtain a fitting for eyeglasses through digital imaging of frames and lenses; and to either concurrently purchases eyeglasses and contact lenses at a diagnostic location or later elsewhere from a PC.

### BEST MODE FOR CARRYING OUT INVENTION

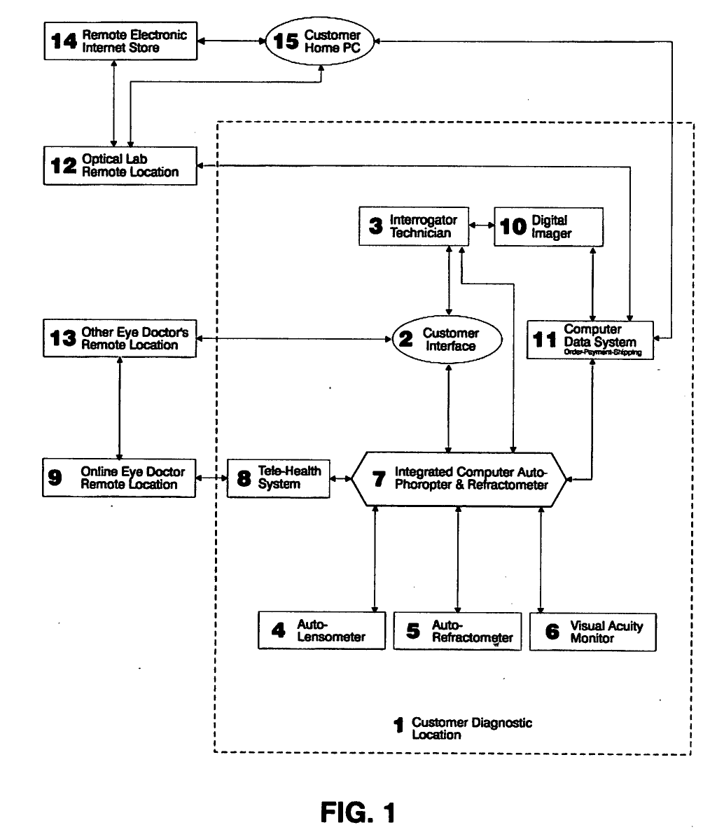

Referring now to flow diagram, FIG. 1, a system and embodiments enabling a customer to purchase eyeglasses and contact lenses from home or some other convenient location includes one or more customer diagnostic locations 1, and a customer interface 2, and each including an interrogator 3, for determining customer order, shipping, and payment contact, and technical fitting data, i.e. pupillary distances, bifocal seg-heights, lens and frame styles and sizes, and a tele-health interface 8 system, to a remote, on-line eye doctor 9;

After a customer 2 cooperates with the interrogator 3, the customer data may be interfaced with the on-line eye doctor 9 who determines the lens specifications or prescription data which is sent back to the electronic diagnostic location 2 and stored at its Computer Data System 11;

The customer 2 may directly interface with an integrated subjective and objective auto-refractometer computer 7 comprising an auto-lensometer 4, auto-refractometer 5 and auto-phoropter integrated subjective and objective refractometer device and computer 7 interfaced with these ophthalmic instruments at the diagnostic location 1 that has a response sensitive, voice-activation system that provides specifications for current or new lens prescriptions for eyeglasses or contact lenses without having to go to a remote on-line electronic eye doctor 9, or an eye doctor elsewhere 13, for an eye examination or refraction;

The interrogator 3 also teaches the customer 2 and performs digital imagery to obtain fitting images 10 in order to allow the customer 2 to browse and store data to purchase frames, lenses, and contact lenses in the computer data system for order, payment and shipping 11. The customer 2 may return to interface at a future date to update the data on the ordering system 11 at the customer diagnostic location 1 or order from a PC 15 directly through the remote electronic internet store 14; The interrogator 3 completes the sales data transaction and from the diagnostic location 1, through its Computer Data System 11, sends the data to the optical lab location 12 for fabrication. The lab 12 either sends the eyeglasses or contact lenses directly to the customer who orders on a PC 15, or to the diagnostic location 1 for customer pick-up.

### More Detailed Description of Drawing

At a later time, the customer 2 may purchase eyeglasses or contacts at the diagnostic center 1 or elsewhere from a P 15, and at a later time, the customer 2 may purchase an exam or obtain an updated prescription either at the diagnostic center 1, through interacting directly with the auto-phoropter system 7, or through the on-line eye doctor 9, or at the place of examination of the remote electronic eye doctor or another eye doctor 13 which provide a quick and convenient method and system to update and purchase vision care services and eyeware glasses or contact lenses product devices and gives the customer choices and alternatives for choosing health care provider eye doctors; the customer may, at a later time, purchase eyeglasses or contacts at the diagnostic center 1, or at a remote electronic interne store 14, either from a customer diagnostic location 1, or from elsewhere on a PC 15

In another aspect of the present invention, customers need not visit diagnostic locations at all. If customers have obtained valid and sufficient specifications for eyeglasses and contact lenses from another eye doctor 13, and enters the corresponding contact and technical data through a PC 15 base to a remote internet, electronic, store location 14 customers may purchase eyeglasses and contact lenses directly and have them shipped either to a convenient store location or to their home or elsewhere 15. In this aspect, customers may visit a convenient diagnostic store 1 and modify or update their data for future refractions, specifications, and orders for eyeglasses and contact lenses at the computer system for order, payment, and shipping 11 from a convenient location 1 or elsewhere on a PC 15.

It is to be understood that the below-described arrangements, interfaces, and systems are only illustrative of the application of the principles of the present invention. In particular the remote electronic store with its remote electronic eye doctor may be accessed using either the Internet or any other communication line, and the customer may connect to the remote electronic store using a PC from any location that happens to be convenient for the customer, not only the customer's home. Furthermore, although the system and method of the present invention are shown with respect to obtaining a vision or sight screening, refraction, or optical lens prescription specifications, and selection to purchase eyeglasses and contact lenses, the system and method can also be used to purchase other optical accessory related products such as contact lens cases, solutions, frame cases and chains, safety glasses, sunglasses, sports glasses, reading glasses, eyeglass repair kits, and warranties.

I have illustrated this with respect to the above with language describing modifications and embodiments however this is not intended to limit these to that language. Several other modifications or arrangements may be devised by those skilled in the art without departing from the spirit and scope of the present invention, and the appended claims are intended to cover such modifications and arrangements.

What is claimed is:

1. A system enabling a customer to obtain a sight screening as well as a fitting and optical prescription for glasses or contact lenses through digital instruments and optical equipment utilized by the customer through voice-activated response, or a technician/optician which provides a virtual refraction and try-on fitting for frames and lenses, and/or an on-line, remote eye doctor (ophthalmologist or optometrist), and information to purchase glasses or contact lenses from a convenient optical store location or at home through a PC, the system comprising:
A) at least one customer diagnostic location to determine a screening for sight and for the optical specifications or prescription using electronic and/or wave-front, refracting ophthalmic instruments, and also to determine a correct fitting for frames and lenses through a virtual try-on, electronic, digital-imager that provides proportionate, to-scale facial, head, eye, and pupil images, and frame and lens color, style, and size choice changes, along with customer ID, data stored on CD, and ordering information to purchase eyeglasses and contact lenses;
B) a remote, on-line eye doctor, who through an interfaced, tele-health system, has means to consult, diagnose, interpret, treat, and transfer health, optical, or medical data and provide results of sight and vision screening, refractive error, and lens prescription and specifications obtained through digital imaging devices, electronic and/or wave-front auto-lensometer, so that the customer may choose and purchase eyeglasses; or, a voice-activated, computerized response self-refracting system unassisted by technician and/or eye doctor. The system interfaces an auto-refractometer and auto-phoropter instruments to provide data to the remote eye doctor to determine specifications for new prescription eyeglasses, and is responsive to said information for providing the customer with the eye doctor's electronic signature and specifications for purchases of prescription optical lenses.
C) without an on-line eye doctor where the customer interacts directly with interfaced auto-refractometer, auto-lensometer, and auto-phoropter instruments through a voice-activated system that drives an integrated, interfaced computer based on the customer's subjective refractive responses and also findings of objective auto-phoropter, auto-lensometer, and auto-refractometer instruments findings, in order to provide specifications for previous specifications for eyeglasses, and/or current refraction or new specifications for eyeglasses or contact lenses, and where the customer interacts with digital imaging devices, with or without an interrogator, so that the customer may choose and purchase eyeglasses and contact lenses, either from a convenient location or elsewhere on a PC.

2. A system for obtaining a sight screening and/or an optical prescription as claimed in claim 1, wherein the customer diagnostic location further comprises:
A) an interrogator/technician/optician for gathering data for the eye doctor who provides the customer's sight screening results and/or optical prescription with electronic signature; and for digitally imaging the customer for virtual try-on, fitting, and choices of frames and lenses, and gathering customer ID data for the purchase of eyeglasses and contact lenses;
B) a customer-direct interface with ophthalmic instruments that provide specifications for previous prescription eyeglasses for duplication of previous eyeglasses, an auto-lensometer, and also direct interface with ophthalmic instruments that provide specifications for current or new eyeglasses, an auto-phoropter which determines an accurate subjective refraction utilizing a voice-activated system based upon the customer's responses, and auto-refractometer, in order to provide specifications for current or new eyeglasses or contact lenses;
C) an interface with the electronic digital imager, electronic and wave-front auto-lensometer and refractometer ophthalmic instruments, and for providing customer ID data and CD ROM;
D) an interface with the remote electronic store providing the customer access through a PC, the results of the sight screening and/or current optical specifications to purchase glasses and contact lenses without having to physically walk-in to the optical store location.

3. A system for purchasing eyeglasses and contact lenses as claimed in claim 2, wherein the optical store further comprises an eyeglasses and contact lens data store for providing optical data; wherein the interrogator/technician has means for providing the results of the sight screening, and eyeglass lenses, frames, and contact lens product information further wherein the customer interface is responsive to the optical product information so as to provide said information to the customer through a PC operated by the customer.

4. A system for purchasing eyeglasses and contact lenses as claimed in claim 3, wherein the optical store, diagnostic location has means for interrogating and receiving eyeglasses and contact lens selection data from the customer and for providing the optical selection data to the remote electronic store; and wherein the remote electronic store technician is responsive to said eyeglasses selection data and suggests to the customer structural material, style characteristics, and sizes of eyeglasses in accord with the selection data and specifications provided by the remote-online eye doctor.
